(19) United States
(12) Patent Application Publication
(10) Pub. No.: US 2022/0088250 A1
(43) Pub. Date: Mar. 24, 2022

(54) TRANSPORTATION DISINFECTION

(71) Applicant: UV Partners, Inc., Grand Haven, MI (US)

(72) Inventor: David W Baarman, Fennville, MI (US)

(21) Appl. No.: 17/418,044

(22) PCT Filed: Dec. 23, 2019
(86) PCT No.: PCT/US19/68291

§ 371 Date: Jun. 24, 2021

Related U.S. Application Data
(60) Provisional application No. 62/789,228, filed on Jan. 7, 2019.

(51) Int. Cl. A61L 2/24; A61L 2/10; A61L 2/26

(52) U.S. Cl. CPC A61L 2/24; A23L 3/28; A61L 2/26; A61L 2/10

(57) ABSTRACT

A disinfecting human interface device with monitoring and safety enhancement system that utilizes user detection and sensors to detect proximity and is designed to provide user feedback on safety, disinfection and decorative lighting. Also having a UV transmissive material designed to enable disinfection of hard to reach surfaces. This disinfecting human interface device has an automatic interlock to protect the user from UV exposure and logs the exposure limits versus exposure. It is connected to a bus system that collects the data and programs the options and enables communications, and potentially to other networks like the cloud.

### FIELD OF THE INVENTION

The present disclosure relates to the field of disinfection, and more particularly toward disinfecting aspects or components of transportation vehicles.

### BACKGROUND

Infection by a foreign organism, such as bacteria, viruses, fungi, or parasites, can be acquired in a variety of ways. But once acquired, the infection, if harmful, may colonize and result in illness. The immune system of the infected host, e.g., the person, may react to the infection and attempt to kill or neutralize the foreign organism. However, in some cases, the immune system may be insufficient to completely neutralize the infection, and hospitalization may be necessary for survival. For these and other reasons, infectious disease prevention is conventionally preferred over reliance solely on the immune system of the infected host.

Conventional efforts to prevent spread of infectious disease often involve manual disinfection techniques, such as wiping down or washing surfaces that may harbor foreign organisms. Because infectious diseases can be spread in a variety of ways, such as via direct contact from person to person, manual disinfection techniques can be time and labor intensive. For example, indirect contact from an infected person to an environmental feature and then to another person who contacts the contaminated environmental feature is a common mode of infection. Because there are numerous surfaces in the environment, it is considered laborious and time intensive to decontaminate all or substantially all surfaces in the environment, essentially making such decontamination impractical in many cases. As another example, air borne pathogens from an infected person can make their way into areas that are inaccessible to manual disinfection techniques.

The transportation environment includes many surfaces that can become contaminated, which can be labor intensive to manually decontaminate due to the number and variety of surfaces (e.g., nooks and crannies as well as various operator and passenger interface surfaces). The duct system in a vehicle is particularly labor intensive to decontaminate, potentially involving removal of the dashboard to access the air ducts. Additionally, or alternatively, in mass transportation environments (e.g., a commuter bus or train), the number and frequency of passengers increases the likelihood of surface contamination, again increasing the labor and time to effectively decontaminate such surfaces with conventional techniques. For these and other reasons, conventional techniques fail to enable decontamination of transportation environments in a practical manner.

In other realms, conventional disinfection systems have involved application of UV light within an enclosed space to protect the user from significant exposure to the UV light. For instance, a target device, such as a dental instrument or surgical instrument, may be stored in a box to protect the user from UV. The mechanical isolation of the target device may be used for safety while over dosing the product with UV light for fast disinfection. Other conventional systems may utilize UV wands and allow portable disinfection by moving the UV wand over a surface. However, disinfection with a UV wand often leads to incomplete disinfection because the dosing amount is substantially controlled by the human operator of the UV wand. Additionally, it is noted that many conventional UV disinfection constructions utilize high intensity application of UV light, which restricts the size of a UV light source to a small or high cost source due to the high power lamps and drivers.

### SUMMARY

The present disclosure in accordance with one embodiment provides enhanced decontamination of surfaces or zones in the transportation environment, potentially yielding better decontamination results over conventional techniques. In one embodiment, movable objects disposed in a transportation environment may be powered and disinfected while tracking touch, temperature or other interface sensors, or any combination thereof, and may enable detecting usage conditions or other conditions pertaining to the transportation environment. For instance, sensors associated with one or more human interfaces in a mass transit system (e.g., a subway car) may facilitate tracking where people load, unload, and usage over a route while providing disinfection as well as data.

In one embodiment, a disinfection system may be provided in conjunction with a human interface device to enable both interface options and disinfection options. The interface options may pertain to disinfection status or aspects in one embodiment; but the present disclosure is not so limited. Human interface devices or capabilities may be incorporated into a variety of embodiments in accordance with one or more embodiments described herein, including switches, cubbies, charging, shifters, floors, air ducts, handles, handholds, touchscreens.

In one embodiment, a movable device can be powered despite its nonstationary configuration (e.g., via wireless power or via a connectionless power link), and can be disinfected by a disinfection system that substantially prevents significant UV exposure to a user or human operator by tracking movement or touch (or one or more other criteria discussed herein) as a basis for turning off the disinfecting device.

One embodiment of the present disclosure facilitates disinfection of disinfection zones in an automated manner By automating the disinfection system, disinfection can be conducted in a controlled and more consistent manner, potentially achieving faster and more effective disinfection.

In one embodiment, the disinfection system may be powered via power from a connectionless power link, such as a wireless power link. The disinfection system may be incorporated into a case or mobile device, potentially facilitating eliminating areas for bacterial and pathogens or other foreign organisms to grow.

In one embodiment, a mobile device may include a battery and a power link (potentially a wireless power link) configured to receive power and transfer that power to charge the battery of the mobile device. In many cases, during charging of the mobile device, it may be held or positioned relative to a surface, such as a positioning structure. Such a positioning structure can hide organisms, such as bacteria and pathogens, or shield surfaces from being disinfected. One embodiment according to the present disclosure may facilitate delivering UV light to such surfaces. For instance, a mechanical and material configuration may enable 3D disinfection. The materials used for the support surface or a case of a mobile device or smart phone can be UV transmissive facilitating delivering UV light to surfaces that otherwise may remain obscured from a conventional system.

In one embodiment, the disinfection system may be configured to utilize UV transmissive elements or materials to facilitate transference of UV light from a remotely positioned UV light source to a disinfection zone or area. Additionally, or alternatively, a control system may be provided with at least one sensor and control operation of the UV light source or deliverance of UV light to the disinfection zone, or both, based on output from the at least one sensor. For instance, the control system may detect a human usage condition, such as presence in proximity to the disinfection zone, based on the sensor output, and discontinue application of UV light to the disinfection zone based on the human usage condition. In this way, the control system may operate in conjunction with the at least one sensor to provide an interlock in an effort to avoid applying substantial amounts of UV light energy to a human.

In one embodiment, UV transmissive materials may be incorporated into one or more surfaces or structures, such as a case for a mobile device or smart phone, that enable treatment in a manner that is considered substantially 3D. For instance, a surface that may be considered a blind surface to conventional disinfection systems may be disinfected in a construction in accordance with one embodiment of the present disclosure.

In one embodiment, via controlled application of UV light energy to a decontamination zone that is conventionally considered a blind zone, intense UV exposure and costly UV light source constructions to achieve such intense exposure may be avoided. The conventional approach of more UV light energy is better may have negative ramifications to the destruction of materials not intended for intense UV exposure. The disinfection system in accordance with one embodiment may substantially avoid such ramifications.

In one embodiment, the disinfection system may incorporate automatic safety monitoring and interlock in conjunction with a user interface that enables user understanding of the process including charge status and disinfection status. This allows automated charging and disinfection while substantially protecting the user and providing a user interface.

These and other objects, advantages, and features of the invention will be more fully understood and appreciated by reference to the description of the current embodiment and the drawings.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components. Any reference to claim elements as “at least one of X, Y and Z” is meant to include any one of X, Y or Z individually, and any combination of X, Y and Z, for example, X, Y, Z; X, Y; X, Z; and Y, Z.

### BRIEF DESCRIPTION OF THE DRAWINGS

FIG. 1 shows a human interface in accordance with one embodiment of the present disclosure in the form of a transmission shifter for a vehicle.

FIG. 2 depicts a movable portion of a human interface in accordance with one embodiment.

FIG. 3 depicts a stationary portion of a human interface in accordance with one embodiment.

FIG. 4 shows a holder for a human interface in accordance with one embodiment.

FIG. 5 shows a human interface in the form of a handhold for a mass transit system in accordance with one embodiment.

FIG. 6 shows an in-vehicle disinfection system for a human interface in accordance with one embodiment.

FIGS. 7A-C depict various views of a human interface in the form of a handle in accordance with one embodiment.

FIG. 8 shows the handle of FIGS. 7A-C in one embodiment.

FIG. 9 depicts an in-vehicle disinfection system for a human interface and vehicle air duct in accordance with one embodiment.

FIG. 10 depicts a representative view of human interface circuitry with the disinfection capabilities in accordance with one embodiment.

FIG. 11 shows a human interface in the form of a door handle for a vehicle in accordance with one embodiment.

### DETAILED DESCRIPTION

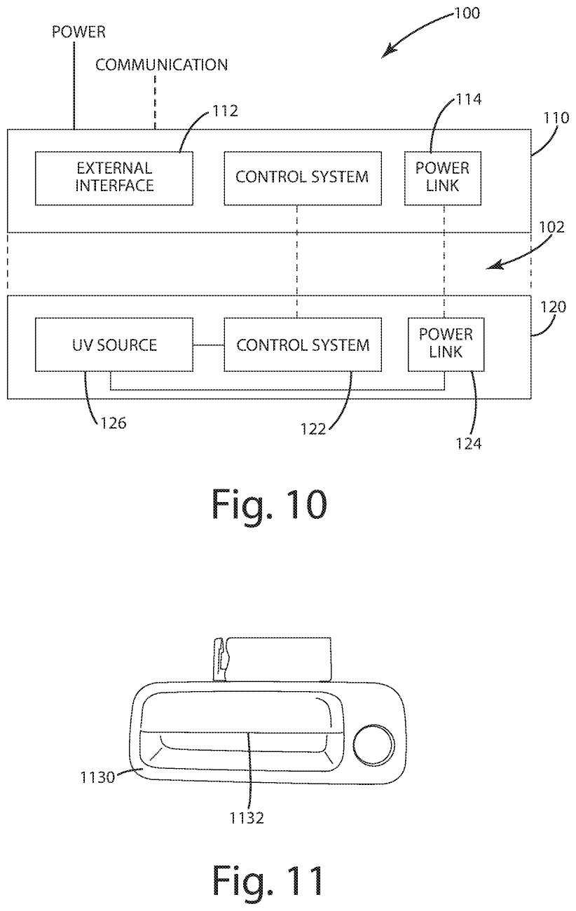

A disinfection system in accordance with one embodiment is provided in the illustrated embodiment of FIG. 10 and designated 100. The disinfection system 100 may include a first portion 110 and a second portion 120, also described herein respectively as a power connection portion 110 and a linked portion 120. In one embodiment, the power connection portion 110 is a stationary portion, and the linked portion 120 is a movable portion; however, the present disclosure is not so limited. In one embodiment, the first portion 110 and the second portion 120 are separable or provided in separate housings. Alternatively, the first portion 110 and the second portion 120 may be integral such that the first portion 110 and the second portion 120 are provided in the same housing or device, as depicted with phantom lines coupling the first and second portions 110, 120.

In the illustrated embodiment, the first portion 110 includes an external interface 112 configured to receive power from an external source. Optionally, the external interface 112 includes a communication interface to facilitate communication with one or more external devices. The first portion 110 may provide a first portion power link 114 operable to supply power to a second portion power link 124 of a second portion 120. Together, the first and second portion power links 114, 124 may form a system power link 102 that facilitates transference of power from the external source to the second portion 120 to power a control system 122 and a UV light source 126 of the disinfection system 100. The system power link 102 in one embodiment may be a connectionless power link, such as a wireless power link.

The second portion 120 or linked portion 120 in the illustrated embodiment includes the control system 122 and the UV light source 126. It should be understood, however, that the present disclosure is not so limited. For instance, the control system 122 or aspects thereof may be disposed in the first portion 110 as depicted in phantom lines in the illustrated embodiment. The UV light source 126, as discussed herein, may be optically coupled to a transmissive element (not shown in FIG. 10) to facilitate deliverance of UV light energy to a disinfection zone.

The control system 122 of the disinfection system 100 may include circuitry operable to direct operation of the UV light source 126, and may include one or more sensors configured to provide sensor information. As an example, the sensor information provided by the one or more sensors may be indicative of a human usage condition with respect to a device, surface, or disinfection zone, or a combination thereof, that is associated with the disinfection system 100. The device or disinfection zone in one embodiment described herein may be a component or associated with such a component in the realm of transportation (e.g., a human interface of a vehicle). The control system 122 may be configured to discontinue application of UV light energy to the disinfection zone in response to the human usage condition being indicative of a human body part being in proximity to or potentially in proximity to the disinfection zone. In one embodiment, the control system 122 may discontinue application of UV light energy within is or less of detecting a human body part in proximity to the disinfection zone.

The control system 122 may be operable to detect or determine an amount of UV dosage (e.g., irradiance mW/m\hat{ }2*exposure times) provided to the disinfection zone and control decontamination of the same based on the amount of UV dosage. Such detection and control may be adaptive based on sensor information indicative of UV dosage to the disinfection zone; alternatively, the detection and control may be estimated based on one or more predetermined parameters (e.g., intensity of the UV light source and transmission effectiveness of the transference element).

The control system 122 may be operable to provide feedback to a human in a variety of ways, indicating a variety of conditions, one or more of which may pertain to decontamination of the disinfection zone. Examples of feedback include haptic feedback, visual feedback, and audio feedback.

To provide a more specific example, the disinfection zone may be associated with a transmission shifter or handhold of a vehicle. The control system 122 may control operation of the UV source 126 to disinfect a human interface portion of the transmission shifter or handhold, and may present visual feedback in the form of light having a spectrum different from the UV light to indicate decontamination is complete or underway. For instance, the visual feedback may be a red light provided to the same transmissive element as the UV light from the UV light source 126, such that the color of the transmissive element as seen by the human is sufficiently distinctive to indicate decontamination is underway.

In one embodiment, the control system 122 may be configured for operator detection. Using motion, acceleration, capacitance touch or power feedback or inputs, the controller may determine a user is present and to shut off UV disinfection.

In one embodiment, the control system 122 may provide motion interlock capabilities and capacitive presence-based capabilities, or both. In one embodiment, motion may be detected using infrared. Additionally, or alternatively, acceleration sensors may provide output indicative of a hand having reached in with respect to the device and indicative of when the device is move, tapped or manipulated. Capacitive proximity sensing may also facilitate identifying touches, and may be provided to the control system 122 as a basis for tracking touches and specific buttons within the plastic and transmissive surfaces of the system.

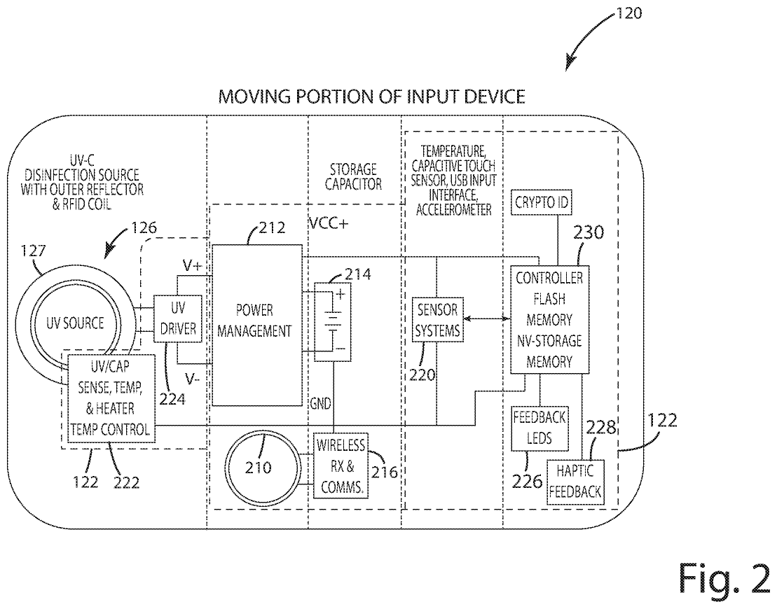

Turning to the illustrated embodiment of FIG. 2, the second portion 120 of the disinfection system 100 is shown in further detail in accordance with one embodiment of the present disclosure. The second portion 120 in the illustrated embodiment includes the second portion power link 124, which may be capable of utilizing power received from the first portion 110 for operation. The second portion power link 124 may include power management circuitry 212 to utilize received power in an efficient manner. The second portion power link 124 in one embodiment may be powered solely from power received inductively by a secondary coil 210. Additionally or alternatively, output from the second portion power link 124 may be augmented through use of a separate power supply, such as a battery 214. The power storage can be sized for dose and interval of typical use. In one embodiment, the second portion power link 124 may provide power for operation based on power obtained solely from the battery 214. The second portion power link 124 may include a capacitor or other conditioning circuitry to regulate power received from the secondary coil 210. In the illustrated embodiment, the second portion power link 124 may be configured to supply a single output for powering all sensor components—but, it should be understood that multiple power domains may be utilized, and that multiple power outputs may be provided dependent on the component ratings, such as a 3.3V for some components and 5V for other components. The second portion power link 124 may also be configured to enable communication (e.g., bidirectional communication) in an effective manner from receiver circuitry 216 via the second portion power link 124. The receiver circuitry 216 may control reception of wireless power and/or communications and optionally transmission of communications. The receiver circuitry 216 may supply power in a regulated manner to the power management circuitry 212 when voltage from a transmission coil for wireless power is present.

The second portion power link 124 may be configured in one embodiment to store power for operation of the UV source 126 for a target dose and interval for decontamination.

The control system 122 in the illustrated embodiment includes a sensor system 220 coupled to one or more sensors 222, including for example a UV sensor, a temperature sensor, a heater/temperature control feedback sensor, an accelerometer, a capacitive touch sensor (e.g. calibrated to touch proximity), and a USB input interface. The control system 122 may also include a controller 230 operable to direct operational aspects of the second portion 110, such as UV dosing and the decontamination process.

The controller 230 includes any and all electrical circuitry and components to carry out the functions and algorithms described herein. Generally speaking, the controller 230 may include one or more microcontrollers, microprocessors, and/or other programmable electronics that are programmed to carry out the functions described herein. The controller 230 may additionally or alternatively include other electronic components that are programmed to carry out the functions described herein, or that support the microcontrollers, microprocessors, and/or other electronics. The other electronic components include, but are not limited to, one or more field programmable gate arrays, systems on a chip, volatile or nonvolatile memory, discrete circuitry, integrated circuits, application specific integrated circuits (ASICs) and/or other hardware, software, or firmware. Such components can be physically configured in any suitable manner, such as by mounting them to one or more circuit boards, or arranging them in other manners, whether combined into a single unit or distributed across multiple units. Such components may be physically distributed in different positions in the disinfection system 100, or they may reside in a common location within disinfection system 100. When physically distributed, the components may communicate using any suitable serial or parallel communication protocol, such as, but not limited to: CAN, LIN, FireWire, I2C, RS-232, RS-422, RS-485, SPI, Ethernet, Universal Serial Bus (USB), RF (cellular, WiFi, Bluetooth, Bluetooth Low Energy.

The controller 230 may include volatile and and/or non-volatile storage memory. For example, the controller may include flash memory. The operation of the controller and related UV disinfection circuitry can be implemented or adapted partially or in full as described in U.S. provisional patent application 62/650,340, entitled Disinfection Behavior Tracking and Ranking, filed on Mar. 30, 2018 to Baarman, which is hereby incorporated by reference in its entirety.

In the illustrated embodiment, the controller 230 may be coupled to a cryptographic identification circuit, which may cryptographically store an identification value associated with the second portion 120.

The control system 122 in the illustrated embodiment of FIG. 2 may include a controller 230 configured for low current operation from a regulated rail, and to monitor temperature (including for example ambient temperature, UV light source temperature, and controller temperature), acceleration, capacitance (e.g., for capacitive touch sensing), and voltage. The control system 122 may also be configured for external communications, configurable decorative lighting detail, and human interface.

More specifically, in one embodiment, the control system 122 may be configured to provide input and output for a human interface, one or more aspects of which may be decontaminated by a decontamination process carried out by the control system 122. For instance, the control system 122 may direct operation of the UV lamp 126 to decontaminate the human interface associated with the second portion 120. It should be understood that the human interface in one embodiment is not limited to a particular set of inputs or outputs, or both. Further, one or more inputs of the human interface may not be directly coupled to the control system 122, such as a transmission shifter of a vehicle providing an input to a system separate from the disinfection system 100 and facilitating directing operation of a vehicle. Likewise, it should be understood that one or more outputs for the human interface may not be directly coupled to the control system 122. In the transmission shifter example, the shifter may include an indication of the current gear selected for the transmission of the vehicle, where such an indication is under control of the system separate from the disinfection system 100.

In the illustrated embodiment, at least one output of the human interface may be directly controlled by the control system 122. Example outputs as described herein, and include but are not limited to feedback via one or more LEDs, potentially of varying colors, and haptic feedback.

In one embodiment, light output from one or more feedback lighting 226 (e.g., LEDs) for output via the human interface may share a transmission channel utilized for disinfection. For instance, the UV light source 126 and one or more feedback lights 226 may direct light to a transmission medium or channel that transfers light to a disinfection zone, which may also serve as a feedback zone for the human interface. Additionally, or alternatively, decorative lighting aspects may be provided to the human interface via the transmission medium. As an example, a decorative LED may be configured in the second portion 120 to direct light to the human interface using the same transmission medium or channel as the UV light source 126.

Although one embodiment of the present disclosure is described in conjunction with feedback lighting 226 or decorative lighting, or both sharing a transmission medium with UV light from the UV light source 126, the present disclosure is not so limited. For instance, the feedback lighting or decorative lighting, or both, may be directed to the human interface with the second portion 120 along a path similar to but not the same as the UV light. The feedback lighting 226 or decorative lighting, or both, may be generated remotely from the human interface and transfer thereto via a transmission medium separate from the transmission medium utilized for transfers of UV light to a disinfection zone of the human interface.

In the illustrated embodiment, the control system 122 of the second portion 120 includes haptic feedback circuitry 228 operable to provide an output to the human interface coupled to or associated with the second portion 120. The haptic feedback circuitry 228 may provide simple or complex patterns of vibration to the human interface that facilitates providing information to the human operator of the human interface.

In the illustrated embodiment of FIG. 2, the UV light source 126 may receive power from a UV driver 224, which may be configured to receive power from the second portion power link 124 under direction of the control system 122. The UV light source 126 may be monitored via a UV sensor 222 by the control system 124 to facilitate control of operation of the UV light source 126 to perform a disinfection procedure. The UV light source 126 may be any type of source capable of generating UV light for disinfection purposes, particularly UVC light. Example constructions of the UV light source 126 include a cold cathode light, a low-pressure Mercury light, or a UV-C LED.

In the illustrated embodiment, the UV light source may include an outer reflector for directing the light generated from the UV light source 126 to a target area, such as a transmission medium or a disinfection zone, or both. In one embodiment, the UV light source 126 may be associated with an RFID coil 127, which may store an identifier associated with the UV light source 126, and potentially one or more usage parameters (e.g., operating time and lifetime).

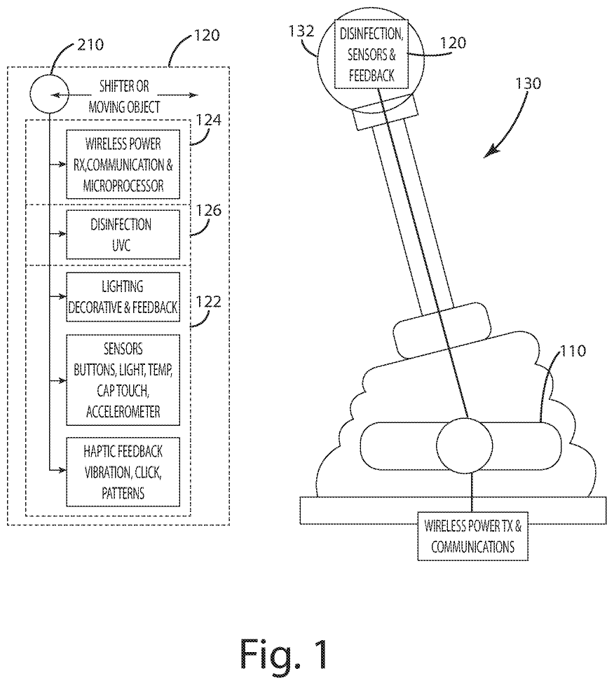

In one embodiment, the second portion 120 depicted in the illustrated embodiment of FIG. 2 may be operable as a sealed shifter knob portion of the human interface for a transmission shifter, as depicted in the illustrated embodiment of FIG. 1. In one embodiment, the second portion 120 may track temperature, UV intensity, touch, or movement, or any combination thereof. The second portion 120 may be powered wirelessly by the power link (e.g., Tx from the first portion 110) and communicate to the first portion 110 (e.g., Tx of the first portion 110) with in band communication. The present disclosure is not limited to in band communication; for example, communication may include other RF solutions, such as BLE. The haptics may be part of the feedback enabling complex patterns and vibration to be implemented in the shifter knob or modular moving and separable user interface. The LEDs or Laser LED through fiber can be used for interface feedback and programmable color and intensity for decorative options.

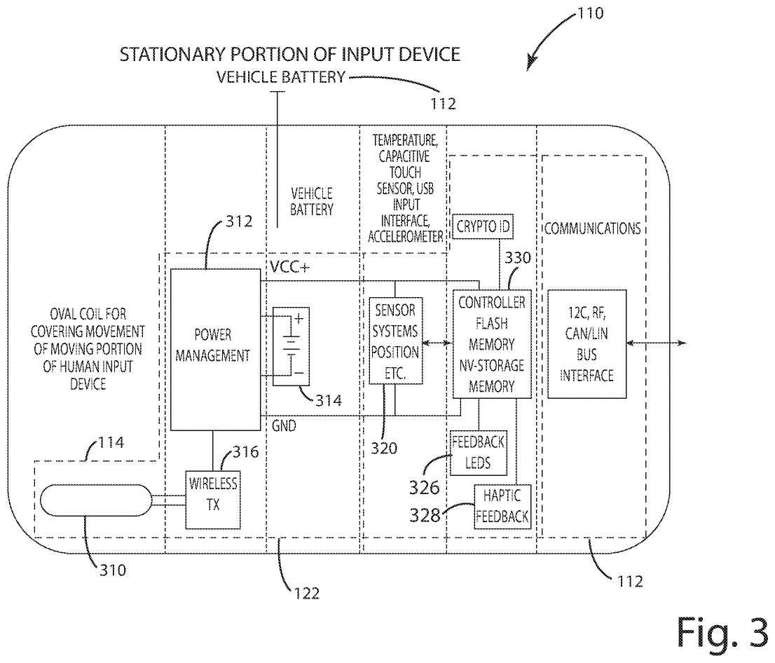

In the illustrated embodiment of FIG. 3, the first portion 110 of the disinfection system 100 is shown in further detail in accordance with one embodiment of the present disclosure. The first portion may include one or more aspects of the control system 122, including a controller 330 configured similar to the controller 230 of the second portion 120. The first portion power link 114 of the first portion 110 may be operable to receive power externally from a vehicle battery or a transportation-based power source and transfer the power wirelessly via the power link 101 to the second portion 120. In this way, the second portion 120 may be provided in a transportation environment without requiring significant redesign to route wiring or other modes of providing power to the second portion 120 while allowing the second portion 120 to move relative to the first portion 110. In one embodiment, the system 100 may form a completely modular disinfection system, and enable feedback, decoration, and allow one or more modules of the system 100 to be sealed for environments. The transmissive materials can be ultrasonically sealed for a water proof seal while still allowing movement.

The first portion 110 may include a sensor system 330 configured similar to the sensor system 220 of the second portion 120, as well as being configured to provide one or more inputs or one or more outputs, or a combination thereof, including for example the feedback circuitry 328 and feedback lighting 326. The control system 122 disposed in the first portion 110 may also include a cryptographic identification circuitry operable to provide secure memory for storing information associated with the first portion 110. In the illustrated embodiment, the external interface 112 is identified by the vehicle battery coupling and communication circuitry designated as 112. As discussed herein, the external interface 112 may facilitate communication with one or more external devices or systems in a variety of ways, including for instance I2C, RF, CAN bus, and LIN bus.

The first portion 110 in the illustrated embodiment of the FIG. 3 is a stationary wireless transmission portion of the disinfection system 100. The first portion 110 may obtain automotive power and condition it for use in transferring the power to the remote portion 120. The geometry of the Tx coil for wireless power transmission may be configured to enable Rx movement and powering effectively, assuring a field is present in all locations of the movable remote portion 120, such as a movable shifter. Additional lighting and feedback options for the human interface device (e.g., shifter) may also be provided at the stationary portion of the human interface device. The CAN and LIN communications interface communicate to the vehicle bus. The crypto device may enable anti-hacking communications security.

Turning to the illustrated embodiment of FIG. 1, the disinfection system 100 is shown in conjunction with a human interface 130 in the form of a transmission shifter for a vehicle. As described herein, the human interface 130 may take different forms, including for example, a vehicle door handle and a handhold for a bus or subway car. The disinfection system 100 may be operable to disinfect one or more surfaces or disinfection zones of the human interface 130.

In the illustrated embodiment, the first portion 110 of the disinfection system 100 may be disposed near a base portion of the human interface 130 that remains substantially stationary or immobile during operation. The second portion 120 of the disinfection system may be disposed in a knob or handhold of the shifter that is movable relative to the stationary portion or first portion 110 but coupled thereto. Alternatively, the movable portion or second portion 120 may be remote or separable from the stationary portion.

The second portion 120 of the disinfection system 100 may be operable to conduct a disinfection or decontamination process with respect to a decontamination zone 132 of the human interface 130. In the illustrated embodiment, the decontamination zone 132 is associated with a portion of the human interface that is determined to be in likely contact with a human body part of the human operator. The disinfection system 100, including the second portion 120, may be constructed to dispose the decontamination zone 132 in optical communication with the UV light source 126. Optical communication may be achieved in a variety of ways, including providing a transmissive medium or transmissive material for transferring UV light from a remotely positioned UV light source 126 relative to the decontamination zone 132. Optical communication may also be facilitated via use of transmissive material that forms one or more aspects of the human interface 132; for example, the transmissive material may form part of a housing of the human interface 130 such that providing UV light from the UV light source 126 to the transmissive material enables decontamination of a surface of the transmissive material.

In the illustrated embodiment of FIG. 1, the second portion 120 may include a second portion power link 124, a disinfection source (e.g., UV light source 126), and a control system 122, similar in many respects to the second portion power link 124, the UV light source 126, and the control system 122 described in conjunction with the illustrated embodiment of FIG. 2. For instance, the second portion power 124 may be coupled to a receiver coil or secondary coil 210 to receive wireless power from the first portion 110 disposed in or proximal to a base of the human interface 130 in the form of the transmission shifter shown in the illustrated embodiment. The control system 122 may be operable to provide feedback lighting 226 and haptic feedback 228 to the shifter knob proximal to the disinfection zone 132. As discussed herein, the present disclosure is not limited to feedback lighting 226 and haptic feedback 228 as an output set for the disinfection zone of the human interface 130. Additional or alternative outputs may be provided.

In the illustrated embodiment, the control system 122 may include decorative lighting aspect as well as the feedback lighting 226. The feedback lighting and/or the decorative lighting may be generated from a light source remote from the disinfection zone 132, but optically communicated or transferred to the disinfection zone 132 or other portions of the human interface 130 via a transmission medium (e.g., a light pipe or fiber, or a combination of different mediums).

The control system 122 may include a sensor system that facilitates obtaining feedback or input from the human operator of the human interface 130 and/or one or more operating conditions of the disinfection system 100, such as temperature, capacitive touch sensing, and acceleration. In one embodiment, the control system 122 may determine that a human operator is proximal to or in contact with the human interface 130 based on output from a capacitive touch sensor disposed proximal to the disinfection zone 132 of the human interface 130. In one embodiment, the capacitive touch sensor may be operable to detect proximity of the human operator relative to the disinfection zone 132 within 10 cm or less, or 1 cm or less. The determination that a human operator is proximal to or in contact with the human interface 130 may be indicative of a human usage condition of the human interface 130 and detected as such.

In one embodiment, the control system 122 may detect changes in acceleration of the second portion 120 or movable portion of the disinfection system 100 based on output from acceleration sensor. Changes in acceleration of the second portion 120, potentially relative to a baseline acceleration of the stationary portion or first portion 110, may be indicative of a human usage condition of the human interface 130.

The control system 122 in the illustrated embodiment may control operation of the disinfection system 100 and a disinfection procedure based on the human usage condition of the human interface 130. For instance, if the control system 122 determines that a human operator is in contact with, proximal to or likely to become in contact with the human interface 130, the control system 122 may discontinue a decontamination procedure of the decontamination zone 132. In this way, the control system 122 may attempt to substantially avoid subjecting the human operator to substantial amounts of UV light energy. In one embodiment, the control system 122 may discontinue a decontamination procedure within a predetermined period of time after detecting a human usage condition with respect to the human interface 130.

It is noted that a human operator may not always be in contact with a human interface 130, such as a transmission shifter of a vehicle, but may be in position to use the human interface 130 intermittently. The control system 122 may receive inputs indicative of the human operator being in such a position although not in contact or substantially proximal to the human interface 130 itself. For example, the control system 122 may receive an input indicative that the human operator is disposed in a driver seat of a vehicle, or that the vehicle itself is mobilized for operation, and the control system 122 may discontinue the disinfection procedure based on such input.

The control system 122 may be operable to initiate a decontamination procedure for the decontamination zone 132 based on one or more inputs, such as proximity of a human operator to the human interface 132. The decontamination procedure may involve subjecting the decontamination zone 132 to UV light in accordance with a UV dosage amount (e.g., irradiance for a period of time). Because the control system 122 is configured to detect the human usage condition, the control system 122 may be operable to determine or conduct a decontamination procedure during times when the human operator is unlikely to utilize the human interface 132. This may allow the control system 122 to conduct a decontamination procedure over a longer period of time, utilizing less irradiance than conventional enclosure-based high-intensity systems, and lessening the likelihood of deterioration or damage to materials surrounding or forming part of the decontamination system 100.

In the illustrated embodiment of FIG. 1, the decontamination system 100 may provide high reliability in delivering power to a moving object while enabling the control and human interface. The lighting control, the sensors, the haptic feedback, and UVC disinfection may be provided. An oval coil or transmitter coil 310 of the first portion 114 allows a smaller coil or receiver coil 210 to move within the field assuring power over the allowable movement and enabling power and interface capabilities to be provided to a previously simple, conventional stick shift knob.

The system 100 in accordance with one embodiment may facilitate powering complex mechanical devices. These devices or human interfaces may be both connected to a vehicle via communications and wireless power. In one embodiment, such devices may include remote moving aspects, which can be powered using Tx coil geometry that enable a range of movement without moving wires or cords to a remote moving Rx, enabling potential avoidance slip rings, cables or contacts and possible reliability issues associated therewith. These devices may be disposed in environments that are challenging for power and human interface while enabling completely the human interface. It should be noted that slip rings and contacts may be a viable alternatively if suitable reliability is achieved.

In one embodiment, when adding power to a free moving shifter knob, the knob may be adapted as a human interface device. Haptic and visual feedback may be provided as a logical interface. Touches, movement, and acceleration patterns may be tracked and monitored, potentially indicating how easy or aggressive the user may be feeling, which may be used as a basis for adjusting operation of the vehicle. The external interface (e.g., CAN and LIN) may enable operation of vehicle functions via user input to the human interface.

In one embodiment, the human interface may be constructed with a second portion 120 adapted to provide heating or cooling surfaces to the decontamination zone of the human interface. Electro-resistive heating, piezo electric heating or cooling, or fans, or a combination thereof may be provided and receive power from the power link 102.

In one embodiment, a human may be detected in proximity to the human interface, and in the context of a transmission shift, the shifter may detect touches and then disinfect between uses or users. The touch sensor and the disinfection in one embodiment may provide a layer of optics and effects capabilities to the human interface.

The disinfection source or UV light source may be configured to disinfect a disinfection zone surface, and then when lighted with an RGB LED, or RGB laser with fiber, the accent color or feedback color can be selected. For example if a car is in park, or if the car is in gear, the system can flash colors or create haptic feedback.

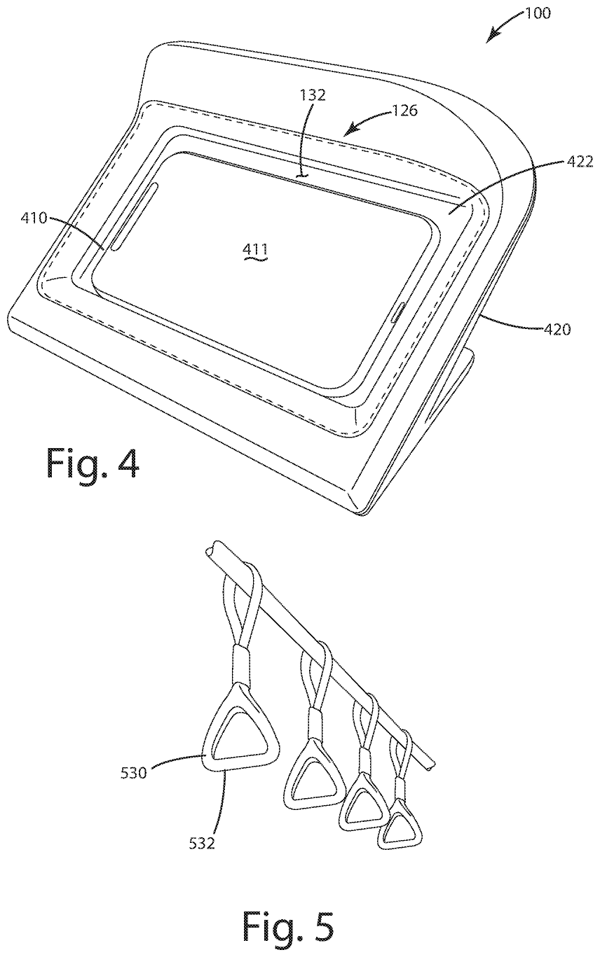

In an alternative embodiment, depicted in the illustrated embodiment of FIG. 4, the decontamination system 100 may be operable to substantially decontaminate one or more decontamination zones 132 of a human interface in the form of a removable portable device 410 and holder 420. The holder 420 in the illustrated embodiment may include aspects of both the first and second portions 110, 120, including a UV light source 126 and a control system 122.

The removable portable device may be a smart phone or other portable device carried by a human operator, and a holder for 20 may be disposed in a transportation system, such as on a transportation bus, and provide charging capabilities for the removable portable device 410. For instance, the holder 420 may include a first portion power link 114 for supplying power in a connectionless manner (e.g. wirelessly or without interconnecting contacts) to the removable portable device 410.

In the illustrated embodiment of FIG. 4, there is a blind surface formed between the holder 420 and the removable portable device 410 that may not be readily subjected to light from an external source. The removable portable device 410, and/or the holder 420 may include a transmissive material capable of being positioned in optical communication with a UV light source 126 (shown in the holder 420). For instance, the holder surface 422 on which the whole portable device 410 is disposed for decontamination may be formed of a UV transmissive material in optical communication with the UV light source 126. The control system 122 may energize the UV light source 126 to transfer UV light energy to the UV treatment in the material of the holder surface 422 subjecting the blind surfaces formed between the holder for 20 and the removable portable device 410 to UV light energy. In this way, a blind surface may form part of a decontamination zone 132 that can be decontaminated by the decontamination system 100.

Additionally, or alternatively, UV light generated from the UV light source 126 may be directed to external surfaces of the removable portable device 410 not directly in contact with the holder 420, such as the screen surface 411 of the removable portable device 410. This may be achieved by constructing the holder 422 to direct UV light via air to portions of the removable portable device 410.

In the illustrated embodiment of FIG. 4, an example of the removable mobile device 410 that can be part of a human interface device 130 connected to a transportation bus via an application and wireless charging is shown. The removable mobile device 410 may be the main display or other devices for video, telematics and other interfaces with disinfection. Application of UV light energy via air may be utilized for UV disinfection while protecting the user.

The illustrated embodiments of FIGS. 5 and 11 depict additional examples of human interface configurations 130. More specifically, in the illustrated embodiment of FIG. 5, a human interface 530 is depicted in the form of a handhold for a transit bus or subway car. The human interface 530 in the illustrated embodiment may be configured in accordance with aspects of the movable portion or second portion 120, with the overhead bar or attachment structure for the human interface 530 being configured in accordance with one or more aspects of the first portion 110. The human interface 530 including the second portion 120 may be powered via the power link 102 (e.g., wirelessly or without a connector). The control system 122 of the second portion 120 may determine whether the human interface 530 is being used by a human (i.e., a human usage condition) and conduct a disinfection procedure with respect to a disinfection zone 532 of the human interface 530. As discussed herein, optional feedback may be provided to the human interface 532 that may indicate one or more conditions to a human operator of the human interface 530. As an example, the human interface 530 may provide haptic feedback to the human operator indicating that the human operator has grabbed a portion of the human interface 530 that is not yet decontaminated. As another example, the human interface 530 including the second portion 120 may provide visual feedback to the human operator indicating the human interface 530 is undergoing a decontamination procedure or has not yet completed a decontamination procedure.

As discussed herein, the human interface 530 may also include decorative lighting under control of the second portion 120, facilitating visually facilitating identification of the human interface 530.

In the illustrated embodiment of FIG. 11, a human interface 1130 is depicted in the form of a door handle for a vehicle. The human interface 1130 in the illustrated embodiment may be configured in accordance with aspects of the disinfection system 100 described herein. In one embodiment, the human interface 1130 may include an integral first and second portions 110, 120 and facilitate disinfection of a decontamination zone 1132. Alternatively, the movable aspect of the handle may be constructed with a second portion 120 in accordance with one embodiment described herein, and powered via a power link 102 with the first portion 110.

The human interface 1130 including the disinfection system 100 may be operable to detect a human usage condition with respect to the human interface 1130, and to conduct decontamination of the decontamination zone 1132 based on the human usage condition. In one embodiment, the disinfection system 100 may receive input indicative of a user approaching the human interface 1130 or in proximity thereto and likely to contact the human interface 1130. If the control system 122 of the disinfection system 100 is currently operating a decontamination procedure, the control system 122 may discontinue the decontamination procedure in response to detecting the human usage condition indicative of the user approaching the human interface 1130 or proximity thereto.

In one embodiment, the human interface 1130 may include one or more light transmissive materials or elements that form aspects of the human interface 1130. For instance, one or more exterior surfaces of the door handle, such as the movable handle itself may be formed of light transmissive material disposed in optical communication with the UV light source 126 of the second portion 120. Activation of the UV light source 126 may be conducted by the control system 122 to disinfect the disinfection zone 1132 of the human interface 1130. The feedback lighting 226 may be provided in a manner that is visible to an approaching user indicating that a decontamination procedure is complete or underway (e.g., green for complete and red for underway). The feedback lighting 226 as described herein may share a common optical path at the UV light source. In one embodiment, the feedback lighting 226 may be directed in a manner that is visible from a distance from the human interface 1130, while the UV light provided to the decontamination zone 1132 may be partially or fully obscured from visibility with respect to an approaching human. Additionally the optical material can be utilized for UV disinfection, decorative details, and user feedback, optionally all with the same optical details.

In the illustrated embodiment of FIGS. 5 and 11, a door handle and a subway handle are shown. For the subway handle, power and data may be distributed above the handle via the handle support. The handle can have temperature and touch monitoring to calculate loading and capacity numbers. When the control system 120 determines that the human interface 530, 1130 is unused, the control system 122 may initiate a disinfection procedure before the next user uses the handle. The feedback lighting 228 can indicate which handles have been disinfected for ease of use. The door handles 1130 may provide the same or similar features, including disinfection, as well as heating for cold weather performance and anti-freezing as well as disinfection and tamper detection. Heating of the human interface 1130, or any other embodiment of a human interface 130 described herein, may be conducted based on sensor feedback obtained by the control system 122 indicative of a temperature of the human interface 130.

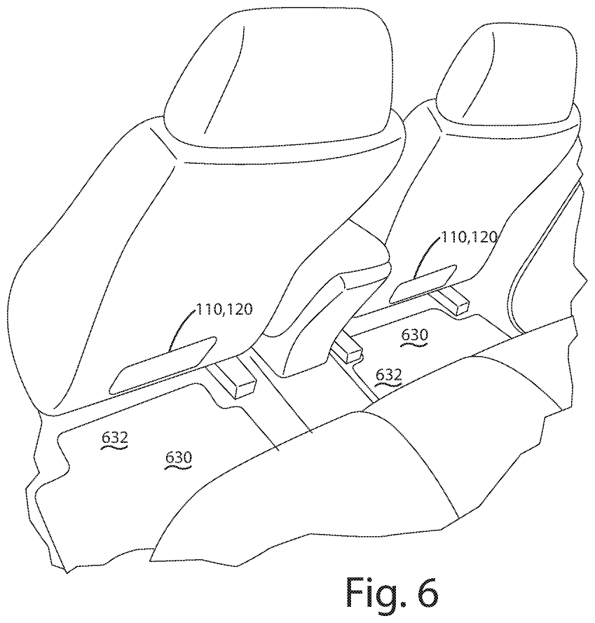

The illustrated embodiment of FIG. 6 depicts a human interface 630 in accordance with one embodiment of the present disclosure in the form of an under seat or under dash disinfection system. The first and second portions 110, 120 may be disposed in a concealed position with respect to a vehicle seat so as to be operable to direct UV light to an area or zone likely to come in contact with a human body part, such as feet or hands of a human operator or passenger. In the illustrated embodiment, the control system 122 of the disinfection system may obtain input indicative of presence of a human in proximity to or within a vehicle cabin of a vehicle and determine a human usage condition accordingly. In one embodiment, the control system 122 may determine presence of a human relative to a plurality of decontamination zones 632 disposed within the vehicle, and control contamination or discontinuance of contamination procedures based on the presence of the human relative to each decontamination zone 632. For instance, decontamination may continue with respect to a first decontamination zone 632 that is determined not to be proximate to the human disposed within the vehicle cabin, whereas decontamination may cease with respect to a second decontamination zone 632 that is determined to be proximate to the human disposed within the vehicle cabin.

In the illustrated embodiment of FIG. 6, the under the seat and under the dash disinfection system 100 may be coupled to an occupancy detector, such as a seat switch to use as a basis for determining to conduct a disinfection procedure when the user is out of the vehicle. The UV dose and time may be set for a minimum dose. In one embodiment, a decontamination procedure or a cleaning cycle may be conducted when the control system 122 determines that the car is parked and the passengers have left the vehicle cabin. This may enable the UV system 100 to dose the areas and create a substantially clean environment for the decontamination zones 632. This may destroy bacteria and in turn the smells and musty odors.

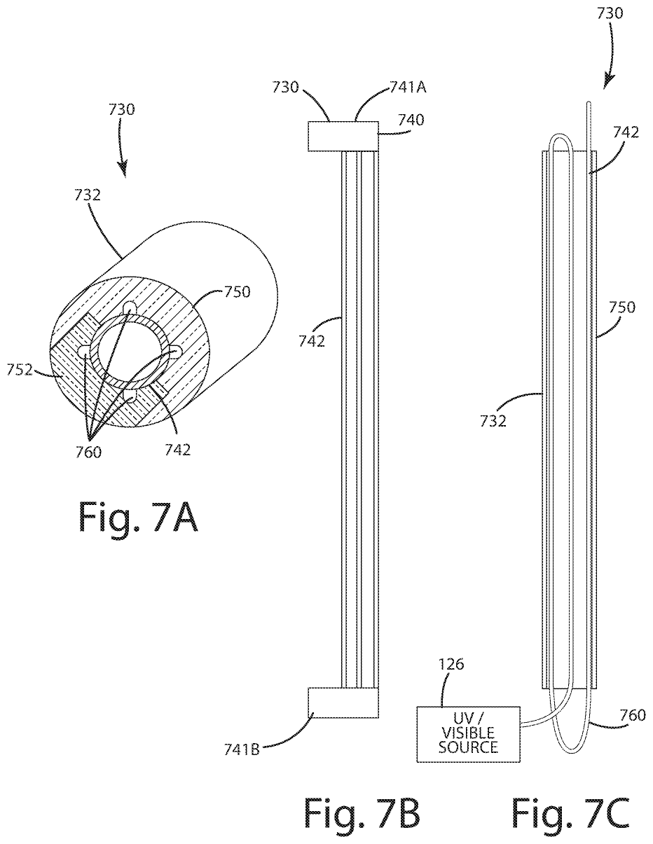

Turning to the illustrated embodiment of FIGS. 7A-7C, a human interface 730 is shown in accordance with one embodiment of the present disclosure. The human interface 730 in the illustrated embodiment is shown without aspects of the second portion 120; and instead the illustrated embodiments of the human interface 730 focus primarily on aspects of the transmission medium for directing UV light or visible light from a remote light source such as the UV light source 126, to a decontamination zone 732. For instance, in the illustrated embodiment, the human interface 730 is shown in the form of a handle for a human to grab or contact, and the decontamination zone 732 corresponds to a region of the human interface 730 that humans are likely to grab or contact. The construction of the human interface 730 may be to facilitate transference of UV light from a light source remotely located from the decontamination zone 732 to the decontamination zone 732.

The human interface 730 may include a base structure 740 constructed to accept and integrate with aspects of the human interface 130 that facilitate use by a human operator and operation of the disinfection system 100. In the illustrated embodiment, the human interface 730 includes first and second ends 741A-B with a support bar 742 disposed therebetween and coupling together the first and second ends 741A-B. A transmission medium 760 may be optically coupled with the UV light source 126, and traverse between the first and second ends 741A-B. The transmission medium 760 may be fiber-optic passages, or light pipes, or any other type of material facilitating transference of UV light energy or visible light energy from one region to another.

In the illustrated embodiment, the transmission medium 760 may include an exterior surface that is generally parallel to a transmission axis for light along the transmission medium 760 from one end to another. The transmission medium 760 may be constructed such that the exterior surface leaks or facilitates emission of light traveling within the transmission medium 760 from the exterior surface. In this way, light, such as UV light from the UV light source 126, may emanate from the exterior surface of the transmission medium 760 along all or a portion of its length. In the illustrated embodiment, the transmission medium 760 may traverse between the first and second ends 741A-B multiple times, enabling emanation of a target level of UV light from the transmission medium 760 along the longitudinal axis of the base structure 740.

In one embodiment, the transmission medium 760 may be optically coupled to an outer interface structure 750, 752 for the human interface 730. Lights, such as UV light, emanating from the transmission medium 760 may traverse through the outer interface structure to an exterior surface of the human interface 730, which corresponds with a decontamination zone 732. With this configuration, in one embodiment, UV light generated from the UV light source 126 remote from the decontamination zone 732 may be optically communicated to the decontamination zone 732 for decontamination thereof in accordance with a decontamination procedure. In the illustrated embodiment, the outer interface structure 750, 752 depicted in the illustrated embodiment is provided in the form of a two piece transmissive wrap or housing with: (a) a first piece 750 where the interface is generally aligned with one longitudinal side of the support 742, and (b) a second piece 752 where the interface is generally aligned with the opposing longitudinal side of the support bar 742, with the first and second pieces 750, 752 interconnecting or interfacing with each other to enclose a longitudinal section of the support bar 742 as well as the transmission medium 760.

In the illustrated embodiment of FIGS. 7A-C, the human interface 730 may take the form of a grab handle for a vehicle train or subway. The human interface 730 may include a first supportive metal grab support for structure as an internal core or support 742. This supportive core may also operate as the capacitive touch electrode. The support 742 may be surrounded by an outer interface 750, 752 constructed as a single extruded wrap or a two piece snap together assembly as shown. Small areas within the outer interface 750, 752 may remain open, and may enable specific dose intervals to allow a fiber to be threaded through the handle. The fiber or transmission medium 760, as described herein, may also be used to facilitate decorative lighting, backlighting, or disinfecting transmissivity, or a combination thereof. In the illustrated embodiment, the transmission medium 760 traverses the longitudinal length of the human interface 730 four times to achieve a target dose for the decontamination zone 732

In one embodiment of the human interface 730, the system may be operable to provide door open feedback with a flashing red light or to vibrate the door handle when a filter needs to be changed. The medium may enable feedback and input for tracking and providing information to the user. In one embodiment, the human interface 730 may be constructed using a quartz fiber with a side projecting PFA casing, providing lighting spiral on a shifter or a wrap for a shifter or handle for lighting effects (potentially unique) while substantially maintaining the UVC effectivity.

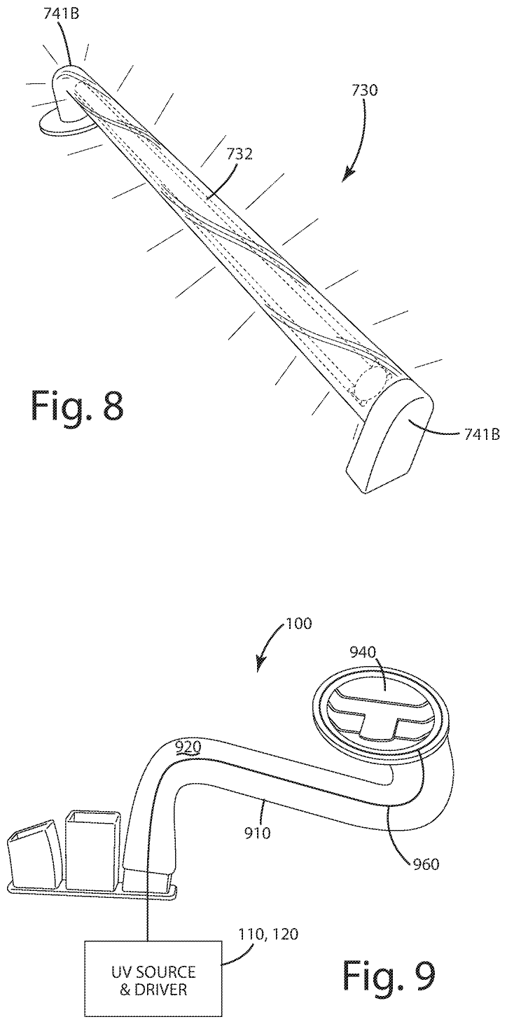

The illustrated embodiment of FIG. 8 depicts the human interface 730 with the UV light source enabled. As discussed herein, the transmission medium 760 of the human interface 730 may also allow an RGB laser diode to set over a million different colors and options for feedback lighting or decorative lighting, or a combination thereof.

The transmissive element 760 or material may take a variety of forms. In one embodiment, the transmissive element 760 is plastic injected PFA for UV-C transmission. Such a plastic is sold under the brand name TEFLON. The thickness of the material may determine the transmissive capability so thinner material selection typically provides greater transmissibility and disinfection capabilities. The inner surface textures allows scattered reflection. The inner surfaces may also be coated with a reflector to protect the device from UV exposure and also provides a dispersion and reflection of the UV light. A textured surface with a reflector provides the best performance. The transmissive element 760 described in conjunction with the illustrated embodiment of FIGS. 7A-C may be incorporated into any of the embodiments described herein.

In one embodiment, inside-out, outside projection, inside-out to transmitter substrate, and outside to transmitter substrate with respect to light may be achieved in a variety of ways, including one or more of the following:
a UV source lighting from above that is directed to the device;
a UV source from below the device transmitting UV through a transmissive material;
a UV source above and below where the UV source on the supporting material transmits UV through the transmissive supporting material allowing the bottom to be disinfected properly with interlocks;
a UV source above where the UV source disinfects the human interface device by first radiating on the device and secondarily transmitting UV through supporting material, transmits UV through the transmissive supporting material allowing the bottom or side to be disinfected properly-dependent on mounting; and
a UV source disinfecting a case around the human interface device wherein the case distributed the UV to areas around the human interface device for proper disinfection.

In one embodiment, a handle as a human interface 730 in accordance with one embodiment may provide a mechanically strong handle that includes a disinfecting surface. The surface may be lighted with decorative lighting and provided with UVC disinfection. The handle may form a human interface device that, as an example, can also track touches, movement, force, and allow feedback-like warnings, error states and arriving stations. For instance, a haptic feedback may be provided to a user indicating that his or her stop is approaching, or that a stop in general is approaching.

In one embodiment, the composition and configuration of the thermoplastic composition or transmissive element and the UV reflective material can be selected to provide a composition with desired levels of UV reflectivity, and transmissivity for a desired application. The composition of the thermoplastic composition or transmissive element may also be selected to be cost-effective, resistant to degradation upon exposure to UV radiation for at least a desired period of time. Utilizing PFA and e-PTFE is a great example of a reflector and UV-C transmissive material. Further details and examples of UV reflective material appropriate for use in the present invention are described in U.S. provisional patent application 62/650,340, entitled Disinfection Behavior Tracking and Ranking, filed on Mar. 30, 2018 to Baarman, and U.S. provisional patent application 62/683,933, entitled Mobile Device Disinfection, filed on Jun. 12, 2018 to Baarman, which are both incorporated by reference in their entirety.

In the illustrated embodiment of FIG. 9, the disinfection system 100 is incorporated into a vehicle in a manner operable to disinfect a disinfection zone 930 that is considered substantially inaccessible to a human operator but provides contamination services and/or modes of infection transportation to the surrounding environment. The disinfection system 100 in the illustrated embodiment of FIG. 9 is associated with a vent system or air duct system of a vehicle; however, the present disclosure is not so limited. Any type of vent system or air duct system may be configured according to one or more embodiments herein to facilitate disinfection thereof or aspects thereof.

In the illustrated embodiment, the first and second portions 110, 120 of the disinfection system 100 are shown in a substantially integrated form with the control system 122 and powerlink 102 to the UV light source 126 integral to a single housing. It should be understood however, that the first and second portions 110, 120 may be configured differently and potentially separate from each other in accordance with one or more embodiments herein.

The disinfection system 100 in the illustrated embodiment of FIG. 9 includes a transmission medium 960 that may traverse through a void 930 or space defined by a vehicle air duct system. The transmission medium 960 in the illustrated embodiment may be optically coupled to the UV light source 126 of the disinfection system 100, and may facilitate emanation of UV light from an exterior surface of the transmission medium 960 to the void 930 of the vehicle air duct system. The transmission medium 960 may be shared by feedback lighting or decorative lighting, or both, to facilitate emanation of visible light to a user to indicate one or more conditions, such as whether a decontamination procedure is underway or complete. Additionally, or alternatively, a second transmission medium (not shown) may follow a similar path after the transmission medium 960, and provide a light path for feedback lighting or decorative lighting, or both.

In the illustrated embodiment, the transmission medium may traverse through the void 932 to an exit point or entry point of the vehicle air duct system, and provide a decorative aspect or feedback aspect to the disinfection system 100. Near the exit point or entry 940, light emanating from the transmission medium may be visible to a user or human operator. As a result, the decorative aspect or feedback aspect of the disinfection system 100 may be capable of relaying information via visible light to the human operator near the entry or exit point 940 of the vehicle air duct system. For instance, the color spectrum of the visible light emanating from the transmission medium 960 near or in proximity to the exit 940 may be indicative of a temperature of the air exiting or traversing through the air duct system.

FIG. 9 shows a portion of a vehicle HVAC duct with a UVC fiber optic cable. The vent may be disinfected and may be become another human interface device enabling temperature feedback by displaying the color of the present temperature. The system may also enable disinfection of the mold and bacteria captured within the vents. The system may be color coordinated to the interior and programmed to any color option for decorative accents around the vents. The options may be programmed for a variety of effects.

In one embodiment, an optical fiber may be provided to disinfect vents and functionally indicate temperature. The disinfection system 100 can also be set to coordinated color options when not disinfecting. When the HVAC is running, the system may indicate to the user what temperature by displaying color as an option. In one embodiment, a channel can be provided to hold the fiber in a specific pattern to provide and maintain a desired design detail.

Directional terms, such as “vertical,” “horizontal,” “top,” “bottom,” “upper,” “lower,” “inner,” “inwardly,” “outer” and “outwardly,” are used to assist in describing the invention based on the orientation of the embodiments shown in the illustrations. The use of directional terms should not be interpreted to limit the invention to any specific orientation(s).

The above description is that of current embodiments of the invention. Various alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular.

The embodiments of the invention in which an exclusive property or privilege is claimed are defined as follows:

1. A disinfecting human interface system, said system comprising:
a UV light source operable to generate UV light;
a transmissive element operably coupled to the UV light source to direct the UV light to a disinfection zone for a disinfection procedure;
a decorative light element;
a feedback element operable to provide feedback to a human; and
a control system operably coupled to the UV light source, said control system configured to discontinue the disinfection procedure based on detection of a human in proximity to the disinfection zone.

2. The disinfecting human interface system of claim 1 wherein the feedback from the feedback element includes at least one of haptic feedback, visual feedback, and audio feedback.

3. The disinfecting human interface system of claim 1 wherein said control system is configured to discontinue the disinfection procedure within 1 s of detection of the human in proximity to the disinfection zone.

4. The disinfecting human interface system of claim 1 wherein the UV light source is disposed below the transmissive element.

5. The disinfecting human interface system of claim 4 wherein the transmissive element is a UV transmissive support.

6. The disinfecting human interface system of claim 1 comprising a wireless link for power and communications to a transportation user interface device.

7. The disinfecting human interface system of claim 1 comprising a wireless moving link of power and communications to a handle.

8. A protective case capable of transmitting UV throughout allowing more complete 3D disinfection of a device:
a control system operable to track usage and disinfection; and
a self-disinfecting shifter nob.

9. The protective case of claim 8 wherein the control system is operable to track usage of the shifter nob.

10. A movable device for human interface, said movable device comprising:
a power link configured to receive power from a power supply separable from the movable device;
a human touchable surface;
a UV light source operable to generate UV light for disinfection of the human touchable surface;
a usage sensor configured to detect a human interface condition relative to the human touchable surface, said usage sensor configured to generate a usage output indicative of the human interface condition;
a control system operably coupled to the UV light source, said control system configured to direct operation of the UV light source to disinfect the human touchable surface based on the usage output from the usage sensor.

11. The movable device of claim 10 wherein the usage sensor is capable of sensing at least one of motion, acceleration, capacitive-based touch, or power status as a basis for determining the human interface condition.

12. The movable device of claim 10 wherein the usage sensor is a proximity sensor operable to detect presence of a human in proximity to the human touchable surface.

13. The movable device of claim 12 wherein the usage sensor is configured to detect the human touching the human touchable surface.

14. The movable device of claim 12 wherein the human touchable surface is a handle.

15. The movable device of claim 10 wherein the usage sensor is operable to detect the human interface condition in response to determining the human is positioned in a manner capable of touching the human touchable surface.

16. The movable device of claim 15 wherein the usage sensor is a pressure activated switch that indicates the human is disposed in a seat proximal to the human touchable surface, and wherein the control system operates the UV light source based on the human being disposed in the seat.

17. The movable device of claim 16 wherein the human touchable surface is a floor on which feet of the human touch while positioned in the seat.

18. The movable device of claim 16 wherein the human touchable surface is a shifter of a vehicle that is proximal to the seat.

19. A disinfection system for a vehicle, said disinfection system comprising:
a UV light source operable to generate UV light;
a UV transmissive element capable of directing UV light output from the UV light source to a disinfection zone of the vehicle, the disinfection zone being remote from said UV light source;
a sensor operable to determine a vehicle status; and
a control system operably coupled to the UV light source, said control system configured to direct operation of the UV light source based on the vehicle status.

20. The disinfection system of claim 19 wherein the vehicle status pertains to a human usage condition of the vehicle.

21. The disinfection system of claim 20 wherein the human usage condition includes at least one of a human being present within the vehicle, the human being exterior to the vehicle and in proximity to the vehicle, and the human touching a handle of the vehicle.

22. The disinfection system of claim 19 wherein the disinfection zone is an air duct of the vehicle, a shifter of the vehicle, a floor area of the vehicle, a door handle of the vehicle, or a grab handle disposed within the vehicle.

23. The disinfection system of claim 19 wherein the disinfection zone is a human touchable surface, and wherein the human touchable surface is integral with the UV transmissive element such that the UV transmissive element directs UV light from the UV light source to the human touchable surface of the UV transmissive element.

24. The disinfection system of claim 19 wherein the UV transmissive element includes a transmission substrate in communication with the UV light source and a projection substrate operable to direct light from the transmission substrate to a surface of the projection substrate.
